(19) United States
(12) Patent Application Publication
(10) Pub. No.: US 2016/0135254 A1
(43) Pub. Date: May 12, 2016

(54) Heating pad for targeted location

(71) Applicant: Jason Somerville, Burlington, WI (US)

(72) Inventor: Jason Somerville, Burlington, WI (US)

(21) Appl. No.: 14/935,854

(22) Filed: Nov. 9, 2015

Related U.S. Application Data
(60) Provisional application No. 62/077,833, filed on Nov. 10, 2014.

(51) Int. Cl. H05B 3/34; A61F 7/03; A61F 7/00; F24J 1/00

(52) U.S. Cl. CPC H05B 3/342; F24J 1/00; A61F 7/03; A61F 7/007; A61F 2007/0078

(57) ABSTRACT

A heating pad for targeted locations employs a conventional single-use exothermic heating element, containing a supersaturated solutions such as sodium acetate. A second version employs an electric heating element. The heating element is secured to a region of the user's body where major blood vessels are located close to the skin surface, such as the inner wrist, inner thigh, side of the neck, or ankle. The heat from the pad's exothermic reaction warms major blood vessels near the skin surface and allows the user's circulatory system to distribute the heat throughout the body.

### CROSS-REFERENCE TO RELATED APPLICATIONS

This application claims the benefit of U.S. Provisional Application No. 62/077,833, filed Nov. 10, 2014, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to therapeutic devices and in particular to a heating pad for targeted locations. The simple application of heat has proven to be therapeutic in resolving many physical ailments, and also makes the patient more comfortable. Even distribution of applied heat has proven to be a problem, however. Parts of the patient's body become too warm while others remain too cold. A heating pad for targeted locations, which warms major blood vessels near the skin surface and allows the patient's circulatory system to distribute the heat, would resolve this problem.

A search of the prior art reveals various therapeutic devices which have been developed to provide heat. None are closely related to the present invention, but several include features which resemble those of the present invention. Each has proven to be less than satisfactory in its own way. The present invention has been developed for the purpose of addressing and resolving these disadvantages. A therapeutic device which provides a heating pad for targeted locations, would resolve these problems.

Heating device for heating a skin surface on partial areas of the human body, U.S. Pat. No. 6,066,164 (filed Mar. 4, 1998), provides a heating device which targets specific areas of the skin. This device does not form a band which can be held in place using friction and band around a body part.

Heat exchange system for body skin, U.S. Pat. No. 4,425,917 (filed May 7, 1981), provides a heat exchange system which provides heating and cooling effects using replaceable thermal cartridges inserted into a fabric holder. The fabric holder being configured to cover a small area of skin such as the brow or neck. This device does not form a band which can be held in place using friction and band around a body part.

Heating band device, U.S. Pat. No. 5,891,189 (filed Aug. 7, 1997), provides a band which provides heat and pressure to sore muscles and sprains. The band provides a heating coil embedded within the band and is powered by a rechargeable battery. The rechargeable battery would not provide steady power through its charge and would have to be replaced or recharged when the battery dies. Additionally, the heating element is not placed within a stretchable band which holds the unit in place.

Neck band for cooling or heating, US Publication No. 2005/0177925 A1 (filed Feb. 12, 2004), provides a neck ring including a gel pack that can be heated or cooled. The gel pack only covers a portion of the banded area as part of the banded area is only covered by the hook and pile fasteners.

These inventions provide heating/cooling devices which only cover a small area of skin. Such devices may not cover the entire afflicted area and are therefore ineffective in certain situations. Other prior art provides ineffective means of providing heat as the power source is not reliable.

### SUMMARY OF THE INVENTION

Accordingly, the present invention is directed to a heating pad for targeted locations. The pad employs a conventional single-use exothermic heating element, containing a supersaturated solutions such as sodium acetate. A second version employs an electric heating element which is powered by plugging into a wall outlet or with batteries. The heating element is secured to a region of the user's body where major blood vessels are located close to the skin surface, such as the inner wrist, inner thigh, side of the neck, or ankle. The heating element is secured using a band which wraps around the entire area and provides heat across the entire banded area. The heat from the pad's exothermic reaction warms major blood vessels near the skin surface and allows the user's circulatory system to distribute the heat throughout the body.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate two embodiments of the invention and, together with the description, serve to explain the principles of the invention.

FIG. 1 is a front perspective view of the first exemplary embodiment with the heating element removed, displaying the chemical heating element 10, the band 12, and the aperture 12A.

FIG. 2 is a side perspective view of the first exemplary embodiment, displaying the heating element 10 and the band 12.

FIG. 3 is a front perspective view of the second exemplary embodiment, displaying the electric heating element 11 and the power input 14.

FIG. 4 is a front perspective view of the second exemplary embodiment, displaying the electric heating element 11, the band 12, the power converter 13, and the power input 14.

FIG. 5 is a front perspective view of eight examples of the first exemplary embodiment worn at various locations on the human body, displaying the bands 12.

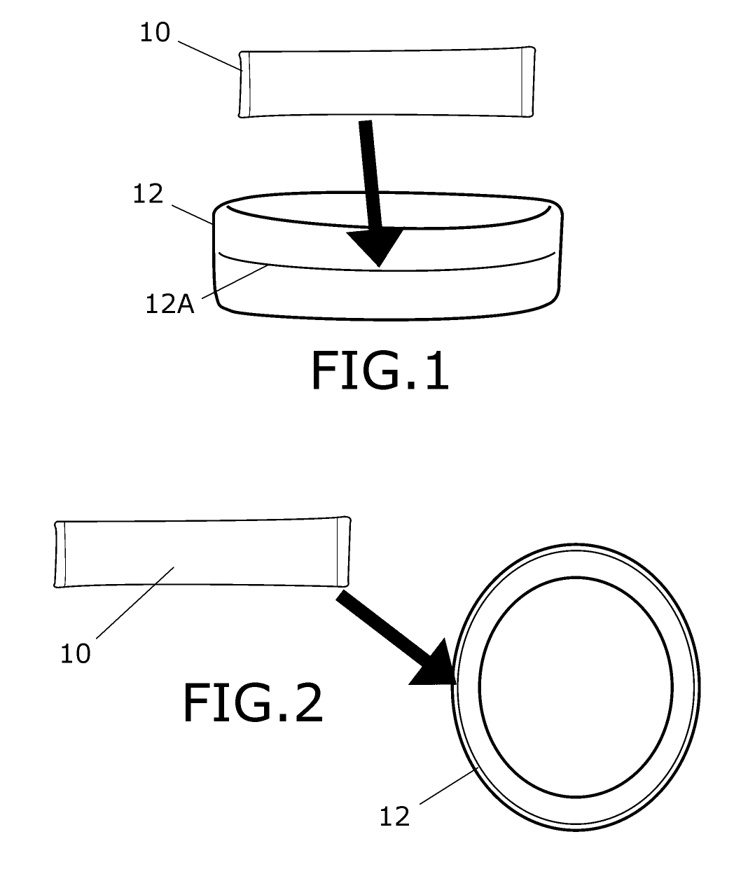

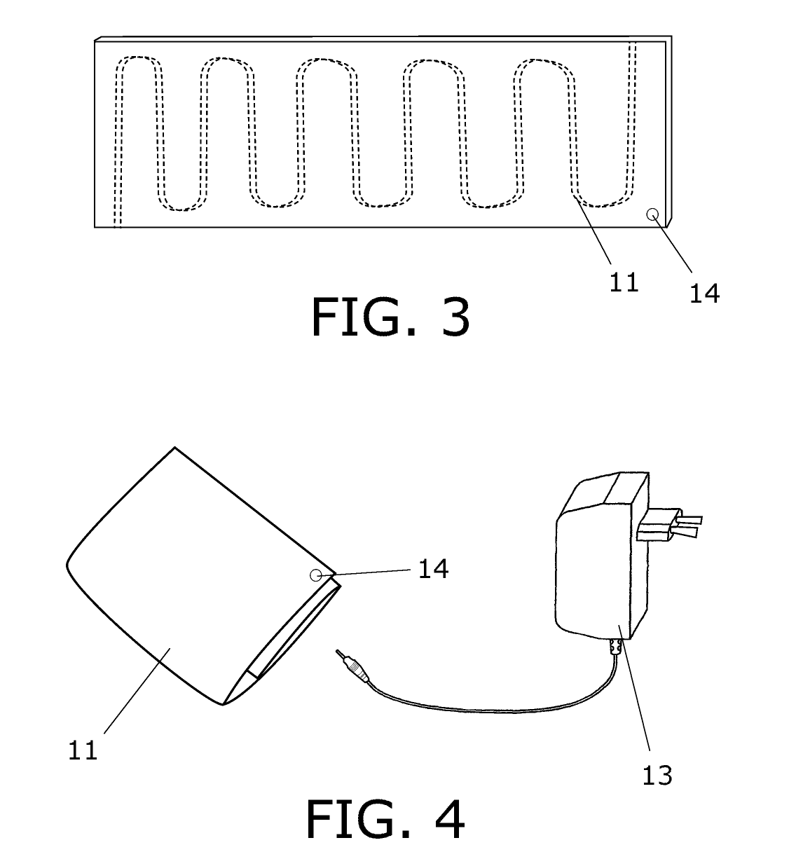

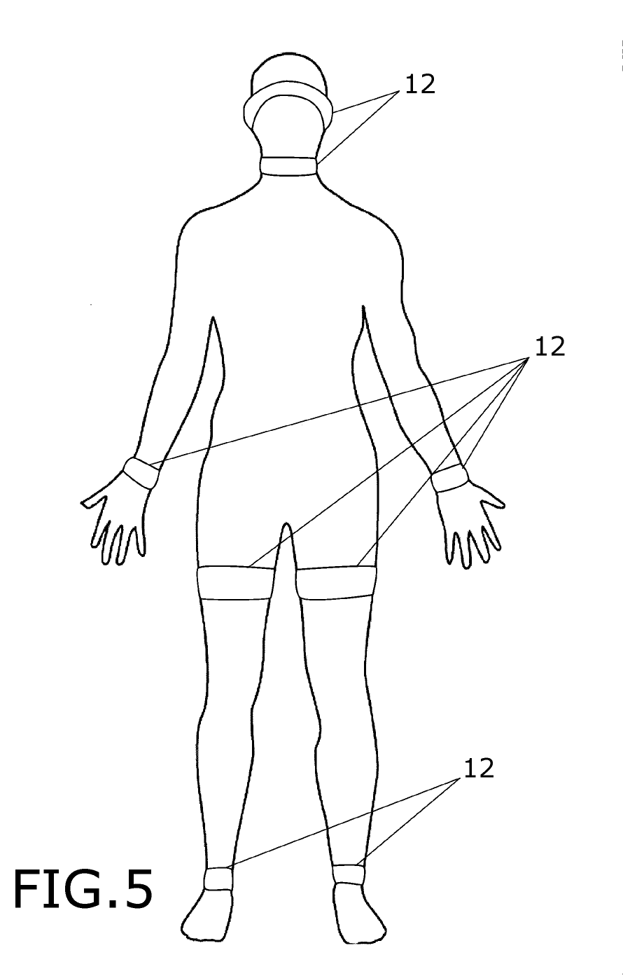

### DETAILED DESCRIPTION OF THE INVENTION

Referring now to the invention in more detail, the invention is directed to a heating pad for targeted locations.

The first exemplary embodiment is comprised of a heating pad which employs a single-use exothermic chemical heating element 10, comprising a supersaturated solution such as sodium acetate. The chemical heating element 10 is enclosed in a stretchable fabric band 12 and secured to a region of the user's body where major blood vessels are located close to the skin surface, such as the inner wrist, inner thigh, side of the neck, or ankle. The band 12 preferably provides a long aperture into which the chemical heating element 10 may be easily inserted, and is provided in a broad range of sizes to fit any of these locations on the human body. The heat from the pad's exothermic reaction warms major blood vessels near the skin surface and allows the user's circulatory system to distribute the heat throughout the body.

To use the first exemplary embodiment, the user activates the chemical heating element 10, inserts it into the band 12 via the aperture 12A, and places the assembled heating pad on a region of the user's body where major blood vessels are located close to the skin surface.

The second exemplary embodiment is similar in structure and function to the first exemplary embodiment, with the following modifications. The chemical heating element 10 is replaced with an electric heating element 11. A removable power converter 13 provides electric power to the electric heating element 11 and may be plugged into any standard wall outlet. Alternatively, the heating element may be powered by batteries—either rechargeable or replaceable.

To use the second exemplary embodiment, the user affixes the power converter 13 to the electric heating element 11, plugs the power converter 13 into a wall outlet, inserts the electric heating element 11 into the band 12 via the aperture 12A, and places the assembled heating pad on a region of the user's body where major blood vessels are located close to the skin surface.

The chemical heating element 10 is preferably manufactured from a flexible, durable material such as plastic, containing a supersaturated solution such as sodium acetate. The electric heating element 11 is preferably manufactured from a flexible, durable material such as plastic, containing a heating coil which is preferably manufactured from a flexible, durable material that conducts electricity, such as copper alloy wire. The band 12 is preferably manufactured from a flexible, durable fabric with an elastic quality, such as elasticized cotton. The power converter 13 is preferably manufactured from braided copper alloy wire sheathed in plastic.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A heating pad for targeted locations, comprising:
a. a single use exothermic chemical heating element;
b. a stretchable fabric band having a long aperture;
c. said long aperture being configured to allow said single use exothermic chemical heating element to be inserted into said stretchable fabric band.

2. A heating pad for targeted locations, comprising:
a. an electrical heating element;
b. said electrical heating element being powered by a power source;
c. said power source being one of rechargeable batteries, replaceable batteries, or a standard wall outlet;
d. a stretchable fabric band having a long aperture;
e. said long aperture being configured to allow said electrical heating element to be inserted into said stretchable fabric band.
